(19) United States
(12) Patent Application Publication
(10) Pub. No.: US 2007/0054261 A1
(43) Pub. Date: Mar. 8, 2007

(54) Methods for retail measurement of skin moisture content

(75) Inventors: Faiz Feisal Sherman, West Chester, OH (US); Vladimir Gartstein, Cincinnati, OH (US); David Burton Moore, Hamilton, OH (US); Carl Hinz Margraf III, Frankfurt Am Main (DE); Brian Keith Fisher, Cincinnati, OH (US); Adam Michael Stamper, Geneva (CH); Colin Kelly, Lowfell (GB); Claire Alexander, London (GB)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/515,141

(22) Filed: Sep. 1, 2006

Related U.S. Application Data
(60) Provisional application No. 60/713,966, filed on Sep. 2, 2005.

(51) Int. Cl. C12Q 1/00; G06Q 50/00

(52) U.S. Cl. 435/4; 705/2

(57) ABSTRACT

Methods provide for measuring scalp moisture content of a consumer. More particularly, the present invention is directed to a method comprising the steps of: (a) collecting input information from said consumer regarding a plurality of characteristics associated with hair and scalp condition of said consumer; (b) providing a moisture meter for measuring said scalp moisture content; (c) using said meter to obtain at least two measured moisture content values from head of said consumer; (d) using said input information collected in step (a) and said measured moisture content values obtained in step (c) to recommend at least one hair care product that will be effective for said consumer in improving health for said hair and scalp of said consumer; and (e) providing said consumer with information regarding said at least one hair care product thereby allowing said consumer to use said at least one hair care product to provide optimum health for said hair and scalp of said consumer.

### CROSS REFERENCE TO RELATED APPLICATIONS

This application claims the benefit of U.S. Provisional application Ser. No. 60/713,966, filed on Sep. 2, 2005.

### FIELD OF THE INVENTION

The present invention relates generally to methods for providing a personal care regimen to a consumer. The present invention also relates to methods for measuring skin moisture content of a consumer. Another aspect of the present invention, relates to methods for measuring scalp moisture content of a consumer in a retail location.

### BACKGROUND OF THE INVENTION

Skin is subject to insults by many extrinsic and intrinsic factors. Extrinsic factors include ultraviolet radiation (e.g., from sun exposure), environmental pollution, wind, heat or infrared radiation (IR), low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. Additionally, the water content of the stratum corneum has a profound influence on the appearance, flexibility, texture, and dryness of the skin, and also on the absorption of drugs and other molecules into and through the skin. The stratum corneum is the outermost layer of the epidermis, and comprises the surface of the skin

Methods of treating the skin generally involve the application of at least one of a variety of appropriate treatments. Such treatments may be selected to provide or to restore certain desired physical or cosmetic characteristics to the skin or scalp. However, unless an appropriate treatment is selected, the desired physical or cosmetic characteristic may not be obtained.

In the case of treating skin, such as the scalp, treatments generally include shampoos, conditioners, colorants, styling compositions, and the like. Manufacturers of these scalp treatments may provide multiple versions of a type or brand of scalp treatment, wherein each of the multiple versions is specifically designed to target a need or demand which is characteristic of a specific consumer segment and which may be based on physical or cosmetic differences of the scalp generally found between respective consumer segments. For example, a single brand shampoo may offer a first version designed to treat flakes and a second version designed to treat dryness, both conditions associated with dandruff.

However, when a consumer is faced with the task of selecting a scalp treatment from among the multiple versions of a scalp care brand, the consumer may unknowingly select a version which is not designed to provide the characteristics desired by the consumer. In such a case, the consumer may be dissatisfied with the results of the selected version of the scalp care brand. As a result of the consumer's dissatisfaction, the consumer subsequently may refuse to select any of the versions of that same scalp care brand even though another version of that scalp care brand may provide the consumer's desired scalp and/or hair characteristics. The occurrence of such circumstances, in turn, may lead to unnecessary loss of sales of the particular scalp care brand for the manufacturer.

Furthermore, retail shopping environments tend to be impersonal. The consumer is left basically alone to pick and choose the appropriate skin or hair care treatments that are most suited to his or her own needs and preferences. Even when a salesperson is available to assist the consumer with or make recommendations for treatment selections, the salesperson's assistance and recommendations are based upon a limited and/or subjective knowledge of the consumer and the consumer's treatment needs.

Methods of measuring moisture content have been developed in the past to determine the moisture level of skin, and have relied on various techniques including resistance and capacitance measurements to obtain the desired indication. However, none of these devices are made integral in the purchasing experience by combining a moisture measuring system with a product line of hair care products.

### SUMMARY OF THE INVENTION

In one embodiment, the present invention is directed to a method for measuring scalp moisture content of a consumer. The method comprises the steps of: (a) collecting input information from the consumer regarding a plurality of characteristics associated with scalp condition of the consumer; (b) providing a moisture meter for measuring the scalp moisture content; (c) using the moisture meter to obtain at least one measured moisture content value from the scalp of the consumer; (d) using the input information and the at least one measured moisture content value to identify at least one hair care product for the consumer; and (e) providing the consumer with information regarding the utilization of the at least one hair care product.

In another embodiment, the present invention is directed to a method for providing a personal care regimen to a consumer for at least one desired benefit toward an improved skin condition. The method comprises the steps of: (a) providing a means for measuring skin moisture content; (b) using the means for measuring skin moisture content to obtain at least two measured moisture content values from the consumer; (c) using the moisture content values to develop the personal care regimen for the consumer; and (d) providing the personal care regimen to the consumer thereby allowing the consumer to use the personal care regimen in a manner to provide optimum health for the skin of the consumer.

In yet another embodiment, the present invention is directed to a method for measuring scalp moisture content of a consumer in a retail location. The method comprises the steps of: (a) requesting input information from a consumer regarding the consumer's hair care preferences, the request of the information being accomplished by questioning means; (b) using a moisture meter to obtain at least one measured moisture content value at a position off the scalp and at least one measured moisture content value at a position on the scalp; (c) analyzing the moisture content values to obtain a scalp moisture value for the scalp; (d) providing the consumer with information regarding the scalp moisture value; and (e) recommending to the consumer at least one product to improve scalp health.

These and other features, aspects and advantages of the present invention will become evident to those skilled in the art from a reading of the present disclosure.

### BRIEF DESCRIPTION OF THE DRAWINGS

While the specification concludes with claims which particularly point out and distinctly claim the invention, it is believed the present invention will be better understood from the following description taken in conjunction with the accompanying drawing.

FIG. 1 is a flow diagram of the process of measuring scalp moisture content of a consumer in accordance with one embodiment of the present invention;

FIGS. 2 and 3 are screen shots of screens which may be used and are examples of block 120 within FIG. 1;

FIGS. 4 and 5 are screen shots of screens which may be used and are examples of block 130 within FIG. 1;

FIGS. 6 and 7 are screen shots of screens which may be used and are examples of block 140 within FIG. 1;

FIG. 8 is a screen shot of a screen which may be used and is an example of block 150 within FIG. 1;

FIGS. 9 and 10 are screen shots of screens which may be used and are examples of block 160 within FIG. 1; and

FIG. 11 is a screen shot of a screen which may be used and is an example of block 170 within FIG. 1.

### DETAILED DESCRIPTION OF THE INVENTION

As used herein, the term “skin” refers to the membranous tissue forming the external covering of a mammalian body including, for example, the external covering of the face, neck, chest, back, arms, hands, legs and scalp.

As used herein, “moisture content” refers to the percentage of water present in the skin.

As used herein, “control area” refers to an area of the skin that is exposed to the environment and convenient for measurement.

As used herein, “test area” refers to the area of skin that a consumer wishes to have tested in order to select an appropriate skin treatment for use by consumer on the area.

According to the present invention, methods are described for measuring the moisture content of scalp and methods of providing a personal care regimen to a consumer. The determination of moisture content in skin, including scalp, is used to quantify various physical and cosmetic characteristics of the skin. For example, a scalp with a low moisture content is unhealthy and may exhibit signs of dandruff, including flakes, dryness, tightness, itchiness and/or redness/irritation. Skin hydration is a function of its normal biological activity that results in continuous moisture flux from within the body to the environment. The improvement of skin barrier function results in greater skin hydration and less moisture loss. As a result, the physical and cosmetic characteristics of the skin may be improved, for example, with treatments that restore skin to normal conditions and improve its barrier function. Improvement of barrier function, in turn, also results in protecting the skin from environmental, physical, chemical, or biological insults and results in an overall improvement in skin health.

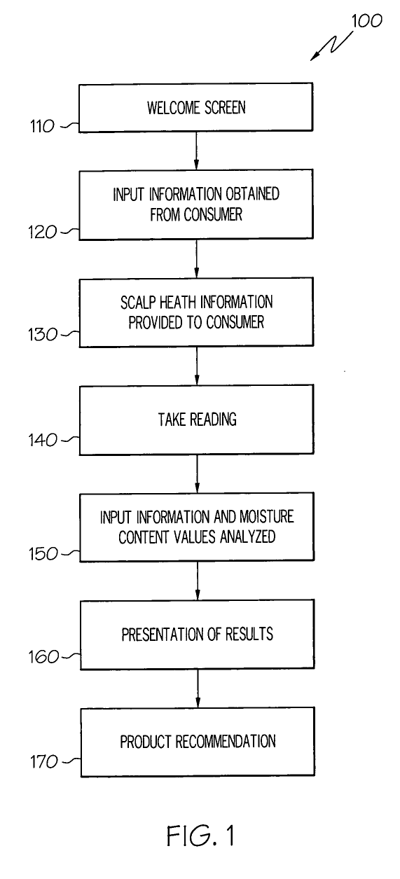

Referring now to FIG. 1, method 100 for measuring scalp moisture content of a consumer comprises the steps of: step 110, welcoming screen; step 120, obtaining input information from consumer; step 130, providing scalp health information to consumer; step 140, taking reading; step 150, analyzing input information and moisture content values; step 160, presenting results to consumer; and step 170, recommending product to consumer. Each of steps 110 to 170 may be represented by screen shots that are intended to give and receive information to a consumer for purposes of executing method 100. These screen shots may be displayed on an imaging device, which may include, but is not limited to, a computer screen, television monitor, light radiating display (for example, PDA), and print materials.

In one embodiment, scalp moisture content measuring method 100 is carried out in a retail location with the aid of a computer system capable of performing automated data analysis for the purpose of analyzing and recommending hair care products, personal care products, and beauty care products, in the form of, lotions, creams, gels, tonics, after shave, sticks, sprays, ointments, pastes, powders, mousse, shampoos, conditioners, oils, colorants, and biomedical and dermatological treatments.

Nonlimiting examples of biomedical and dermatological treatments include prescription skin care treatments, laser treatment, chemical peel, dermabrasion, electrical stimulation, botox treatments, surgical treatments and exfoliating pads and cloths.

In one example, the automated data analysis may be performed using software. Off-the-shelf software may be used. In another example, the software may be modified to meet the specific needs of the particular system and/or user. As used herein, “retail location” includes business establishments stocked with items for sale or with standard services for sale, where consumers go to examine goods and services with the possible intent to buy for their personal or household use. Examples of such retail shopping environments include, without limitation, department stores, shopping malls, shopping centers, open air markets, kiosks, drug stores, mass merchandisers, specialty shops, grocery stores, hair care salons, and convenience stores. Typically, consumers may come and go freely during the normal operating business hours of these retail shopping environments. However, persons skilled in the art would readily appreciate that the method may be used in a variety of other locations without departing from the scope of the present invention. For example, the method could be used in salons, barber shops, doctors' offices, dermatologists' offices, at home, clinical research centers, hospitals, laboratories and public areas.

In one embodiment of the present invention, step 110 contains a welcome screen which identifies the system and the associated hair care products, for example, HEAD & SHOULDERS® products.

Still referring to FIG. 1, step 120 depicts the step of collecting input information from a consumer. In one embodiment, input information may be collected from a consumer by questioning means, that is, by the consumer's answering questions, which are asked of him or her, either orally or in written form, or electronically, such as via a computer terminal or other imaging device. The questioning means may be an interviewer asking oral questions of the consumer, for example, an in-store interview, a written questionnaire on which the consumer writes answers to the written questions, or an electronic questionnaire viewed by the consumer on a computer screen or other imaging device and for which the consumer submits answers to the questions by typing on a keyboard, touching a responsive screen, speaking an answer, or the like. In another embodiment, the information may be collected from a consumer through the use of an interactive site via the internet. The consumer's response to the questions may then be used to assist in recommending an appropriate hair care product for the consumer. These questions may be focused on a plurality of hair and scalp characteristics and/or preferences, including, for example, hair care habits, hair care product use preferences, skin and scalp sensitivity, skin and scalp problems and concerns, and hair care questions.

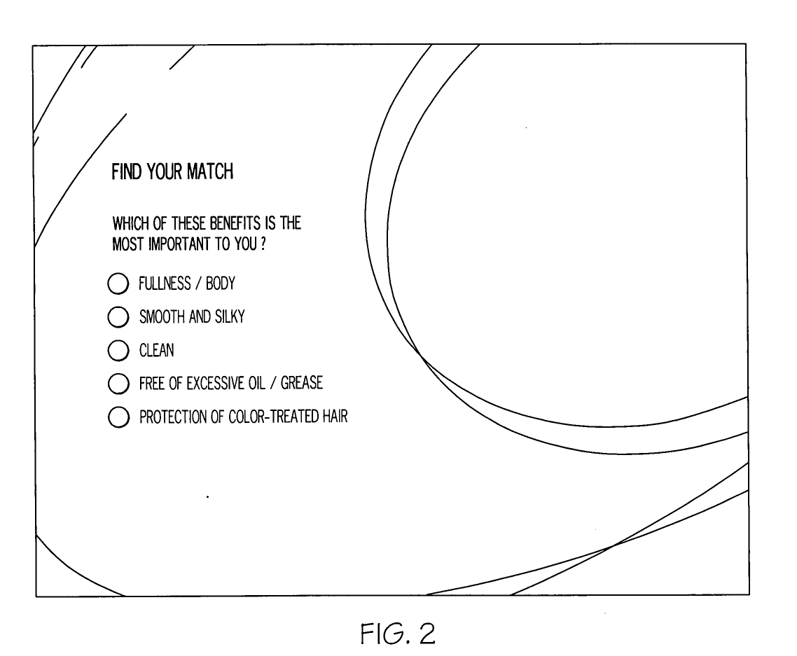

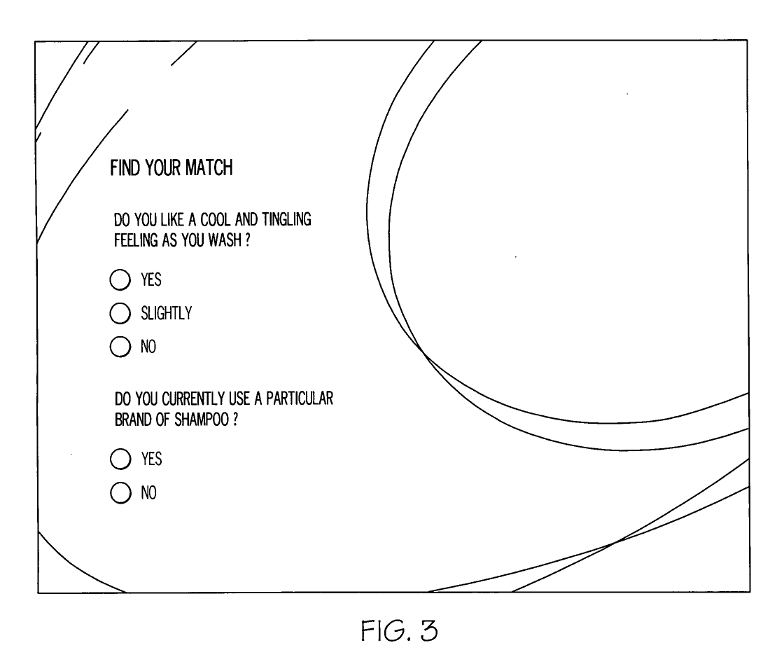

Referring to FIGS. 2 and 3, scalp moisture content measuring method 100 contains step 120. Generally illustrated is text in the form of questions to the consumer such as: Which of these benefits is the most important to you?; Do you like a cool and tingling feeling as you wash?; Do you currently use a particular brand of hair care product? The intent of step 120 is for the purpose of assisting in the making of proper hair care recommendations. The precise manner and wording chosen to collect this information from the consumer may vary depending on local custom, the comfort level of consumers in discussing personal care characteristics, for example, skin, hair and scalp, and the meaning associated with terms which may be used in different parts of the world to collect information desired. It is to be further understood that the methods of the present invention are not to be limited to any one type of question asking methodology or philosophy.

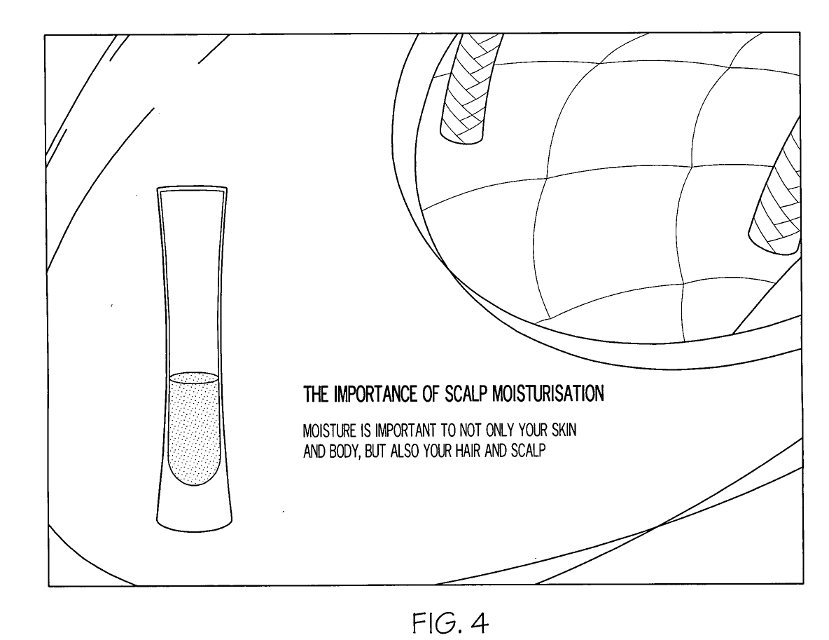

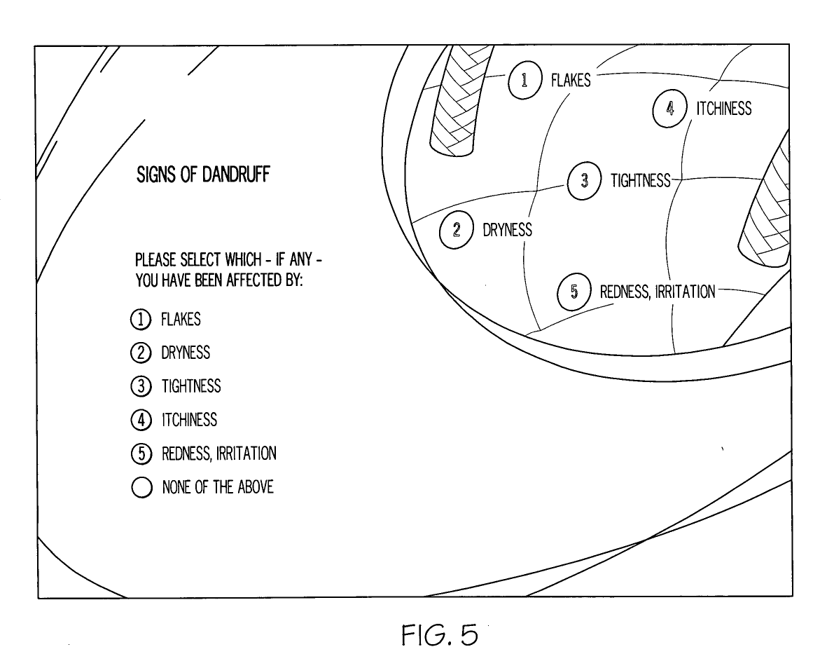

Referring to FIGS. 4 and 5, scalp moisture content measuring method 100 may further contain step 130, that is, providing a consumer with educational information regarding scalp health, for example, scalp moisturization and signs of dandruff. Referring now to FIG. 4 wherein the consumer views content regarding the importance of scalp moisturization. Generally illustrated in FIG. 4 is a close-up or magnified depiction of a segment of human scalp, including hair follicles extending therefrom. Text may be included in this illustration as a warning or caution to alert the user of the need to assure proper moisturization of the scalp. Referring now to FIG. 5 wherein the consumer views content regarding the signs of dandruff. Generally illustrated in FIG. 5 is a close-up or magnified depiction of a segment of human scalp, including hair follicles extending therefrom. Text may be included in this illustration, for example, asking the consumer if they have been affected by certain dandruff conditions, such as, “flakes, dryness, tightness, itchiness, redness, irritation, none of the above.” One skilled in the art will readily appreciate that any words which represent dandruff conditions may be used in this invention.

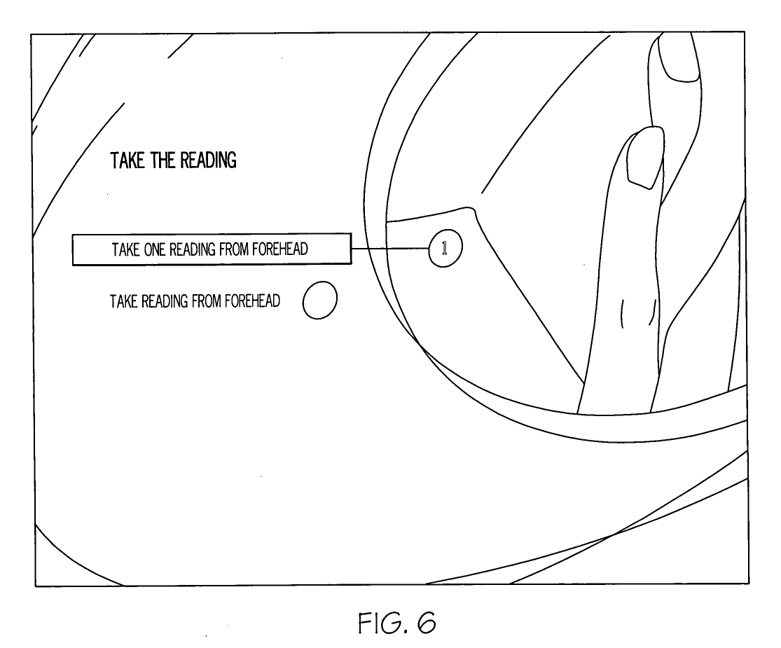

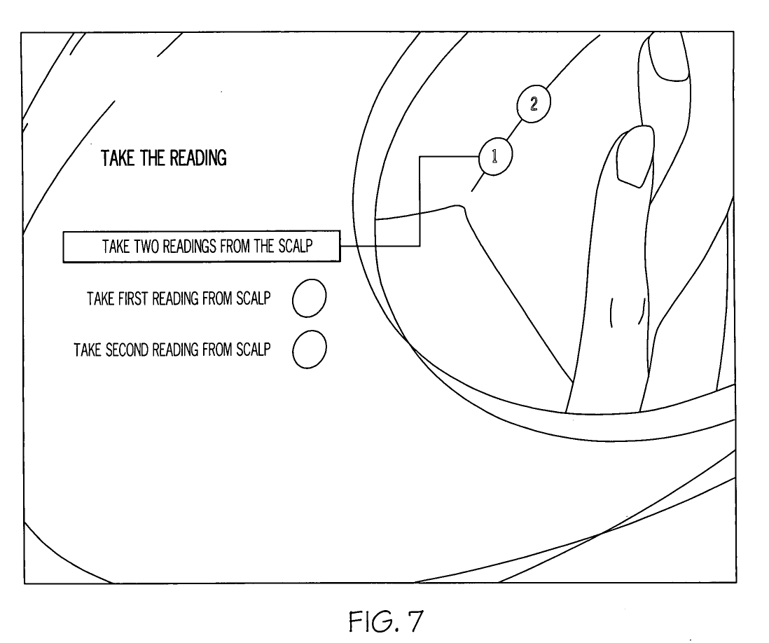

Referring to FIGS. 6 and 7, scalp moisture content measuring method 100 contains step 140, that is, taking/obtaining at least one moisture content value from a position on the scalp of a consumer using a moisture meter for measuring scalp moisture content. Generally illustrated in FIGS. 6 and 7 is a close-up or magnified depiction of the head of a consumer, including the forehead and scalp portions. Text may be included in these illustrations as a prompt to instruct the consumer/in-store consultant to take measurements. Step 140 may be performed by the consumer themselves, or it may be performed on the consumer by another, for example, a beauty counselor or hair care consultant. In one embodiment, the moisture meter may be an electronic device comprising an impedance sensor, as described in U.S. Patent Application entitled Method and Device for Indicating Moisture Content of Skin” filed on Sep. 2, 2005, Attorney Docket No. 10121P. In one embodiment, the consumer is prompted to take/obtain two measured moisture content values, for example, a first measured moisture content value at a position off the scalp of a consumer and a second measured moisture content value at a position on the scalp. In one example, the off the scalp location may include, for example, forehead skin, cheek, chin, ear and arm. With respect to the at least second measured moisture value, several such measurements at various positions on the scalp can be produced. In one example, the moisture meter for measuring scalp moisture content is used to obtain one or more additional measured moisture content values at various locations on the scalp. As each measured moisture content value is captured, the values are displayed in a suitable manner, for example, on an LED display on the moisture meter. In one example, the measured moisture content values are stored in a programmable integrated circuit.

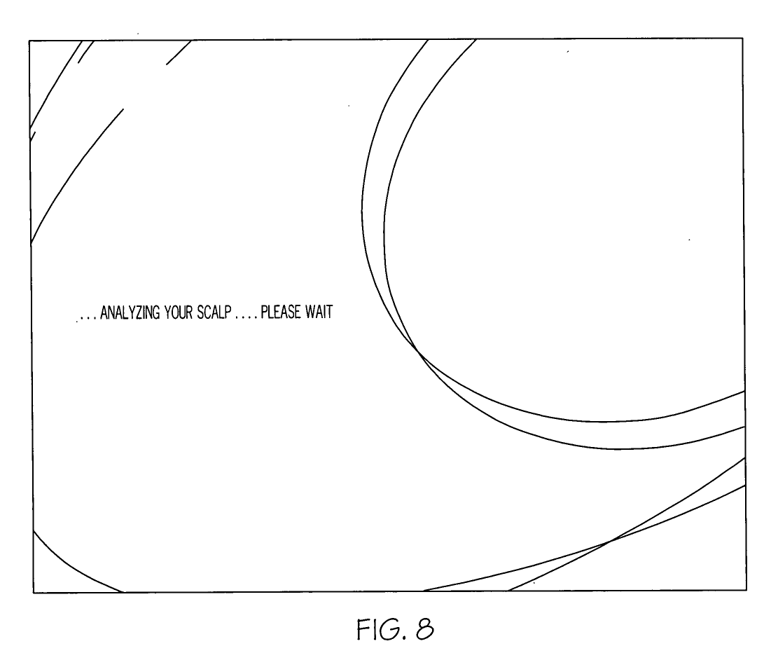

After having collected the input information and measured moisture content values from a consumer as described in steps 120 to 140, scalp moisture content measuring method 100 analyzes this information and corresponding values as depicted in step 150. Referring to FIG. 8, the collected input information and measured moisture content values are analyzed, for example, the first measured moisture content value is compared with the second measured moisture content value to obtain a measured moisture content differential. Generally illustrated is text advising the consumer that the analysis is being performed. In one example, the scalp moisture content value for the position off the scalp (m1) is compared to the scalp moisture content value for the position on the scalp (m2), to obtain a measured moisture content differential (m2−m1) or (m1−m2). In one embodiment, the moisture meter is electrically coupled through a cable to a processing system, such as a conventional PC or laptop computer. In another embodiment, the moisture meter is electrically coupled to a programmable integrated circuit. In yet another embodiment, the moisture meter is electrically coupled to a computer wirelessly. The processing system is operable to convert the measured moisture content values generated by the meter into a measured moisture content differential. In another embodiment, the measured moisture content differential may be determined manually using the formula (m2−m1) or (m1−m2).

If additional scalp moisture values are obtained at positions on the scalp, all of these values are compared to one another and the measurement with the largest value, or lowest value or mean value is then compared with the moisture content value for the position off the scalp. The result of this comparison is a measured moisture content differential profile. In one embodiment, a measurement differential from about 0 MHz to about 0.05 MHz, represents a healthy scalp condition (high moisture level); a measurement differential from about 0.05 MHz to about 0.5 MHz represents a moderate scalp condition (medium moisture level); and a measurement differential greater than about 0.5 MHz represents an unhealthy scalp condition (low moisture level).

Still referring to FIG. 8, the moisture content differential is correlated to a scalp moisture value. In one embodiment, the processing system described above is operable to convert the frequency differential into a scalp moisture value as shown in table I.

TABLE I

| Measured Signal | | |
|---|---|---|
| Difference between m1 | | |
| and m2 (MHz) | Scalp Moisture Value | Scalp Condition |
| >=0.9 | 5 | D |
| 0.79-0.89 | 10 | D |
| 0.68-0.78 | 20 | D |
| 0.54-0.67 | 30 | D |
| 0.43-0.53 | 40 | D |
| 0.34-0.42 | 50 | M |
| 0.25-0.33 | 60 | M |
| 0.15-0.24 | 70 | M |
| 0.06-0.14 | 80 | M |
| 0.04-0.05 | 90 | H |
| <=0.03 | 95 | H |

In one embodiment, the scalp moisture value is from about 0 to about 99, and in another embodiment, from about 5 to about 95. Such a value is based upon the relative differences between the measured values for the off the scalp position and the on the scalp position. As such, the scalp serves as its own control. The scalp moisture value may also be represented by such terms as “healthy (H)”, “moderate (M)”, and “dry (D),” however, any words or a numbered grade scale which depict increasing or decreasing quantities of scalp moisture levels may be used in the invention.

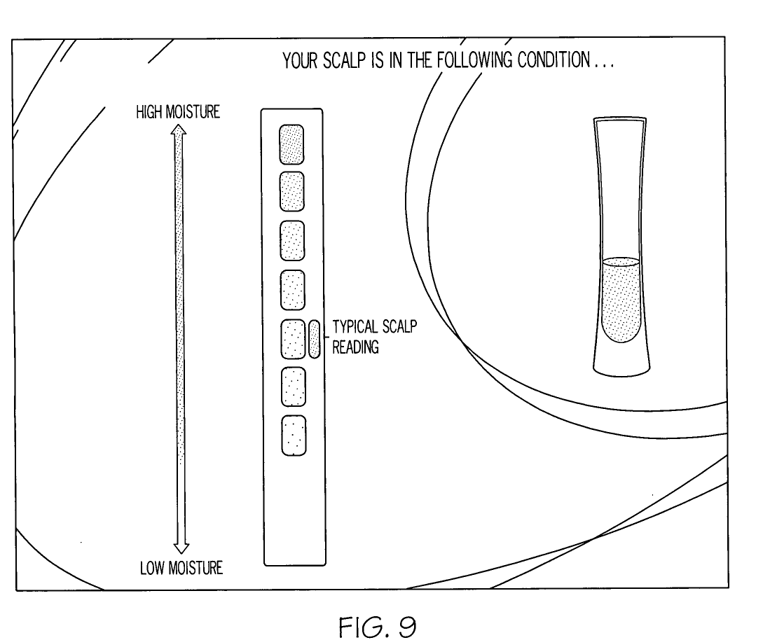

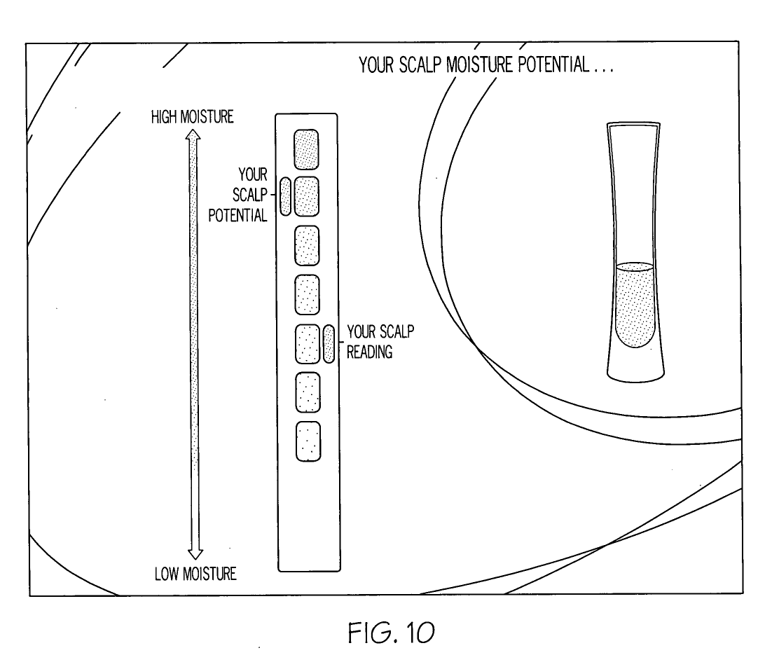

Referring to FIGS. 9 and 10, scalp moisture content measuring method 100 may further contain step 160, that is, giving the results of the scalp moisture content measurements to the consumer. Referring now to FIG. 9 wherein the consumer views content regarding the moisture content of a “typical scalp”. Generally illustrated in FIG. 9 is colored water being poured into a measuring jar up to a certain level that corresponds to a scalp moisture level. Text may be included in this illustration to depict a range of moisture levels from low to high. Referring to FIG. 10 wherein the consumer views content regarding their own scalp moisture content level and their scalp moisture potential. Generally illustrated in FIG. 10 is colored water being poured into a measuring jar up to a certain level that corresponds to the consumer's scalp moisture level. Text may be included in this illustration to depict a range of moisture values from low to high.

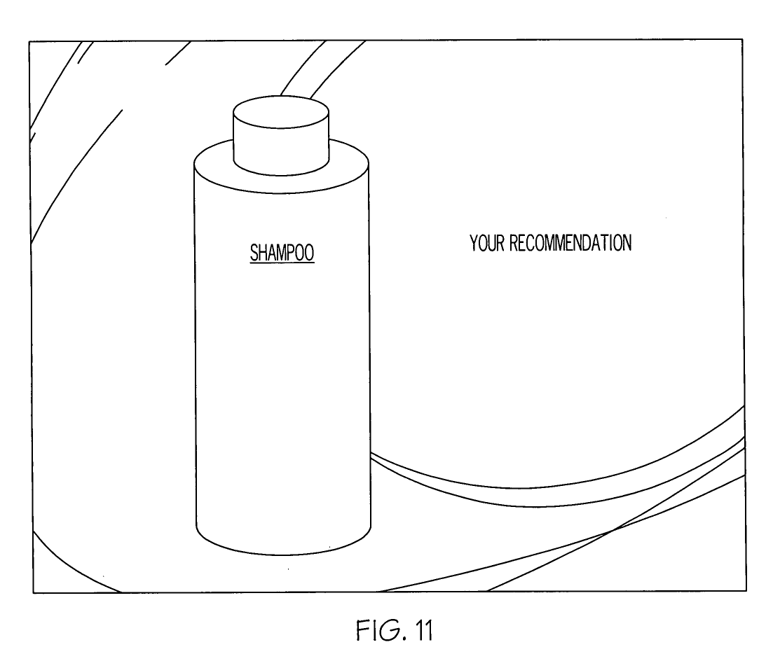

After having analyzed the input information and measured moisture content values from a consumer as described above, step 170 of scalp moisture content measuring method 100 calls for recommending at least one hair care product, thereby providing feedback information to the consumer identifying hair care products that will be effective in improving hair and scalp health. Referring to FIG. 11, wherein the consumer views content regarding a particular hair care product recommendation to a consumer. Generally illustrated is a depiction of a hair care product, for example, HEAD & SHOULDERS® extra volume shampoo. In one example, the product recommendation may be provided to the consumer in the form of, for example, a written report, print-out, or graphical interface outputted to an imaging device. In another example, the product recommendation may be provided to the consumer through a counseling session with a beauty counselor or hair care consultant. Exemplary hair care products may include, without limitation, shampoos, conditioners, oils, colorants, and styling compositions.

Step 170 of the present invention may further comprise recommending more than one hair care product to a consumer. For example, steps 120 to 150 may indicate that a consumer has a dry scalp and as a result, one hair care product is recommended. In another example, steps 120 to 150 may indicate that a consumer has a healthy scalp. As a result, a hybrid recommendation may be made, for example, a hair care product that helps maintain the consumer's healthy condition is recommended along with a hair care product that will help treat scalp concerns if they arise in the future.

Various embodiments of the present invention may be implemented, for example, by operating a computer system to execute a sequence of machine-readable instructions. These instructions may reside in various types of signal bearing media, such as hard drive, flash card, portable memory chip, and main memory. In this respect, another aspect of the present invention concerns a program product, comprising signal bearing media embodying a program of machine-readable instructions, executable by a digital data processor, such as a central processing unit (CPU), to perform method steps. The machine-readable instructions may comprise any one of a number of programming languages known in the art (e.g., Visual Basic, C, C++, etc.).

The CPU may be, for example, a Pentium, 200 mega hertz plus. However, it should be understood that the present invention is not limited to any one make of processor and the invention may be practiced using some other type of a processor such as a coprocessor or an auxiliary processor. An auxiliary storage adapter may be used to connect mass storage devices (such as a hard disk drive) to a computer system. The program need not necessarily all simultaneously reside on the computer system. Indeed, this latter scenario would likely be the case if computer system were a network computer, and therefore, be dependent upon an on-demand shipping mechanism for access to mechanisms or portions of mechanisms that resided on a server. A display adapter may be used to directly connect a display/imaging device to the computer system. A network adapter may be used to connect the computer system to other computer systems.

It is important to note that while the present invention has been described in the context of a fully functional computer system, those skilled in the art will appreciate that the mechanisms of the present invention are capable of being distributed as a program product in a variety of forms, and that the present invention applies equally regardless of the particular type of signal bearing media used to actually carry out the distribution. Examples of signal bearing media include: recordable type media, such as floppy disks, hard disk drives, and CD ROMs and transmission type media, such as digital and analog communications links and wireless devices.

In another embodiment, the present invention is directed to a method of providing a personal care regimen to a consumer, comprising the steps of: (a) providing a means for measuring skin moisture content; (b) using said means for measuring skin moisture content to obtain at least two measured moisture content values from said consumer; (c) using said moisture content values obtained in step (b) to develop said personal care regimen for said consumer; and (d) providing said personal care regimen to said consumer thereby allowing said consumer to use said personal care regimen in a manner to provide optimum health for said skin of said consumer. In one example, the method of the present invention is performed in a retail location.

In one embodiment, any suitable means for measuring skin moisture content may be used. For example, an electronic device comprising an impedance sensor, as described above, can be used to measure the moisture content of skin. In still other embodiments, suitable means for measuring moisture of skin also include radio frequency, infrared, nuclear magnetic resonance, mechanical vibration, skin deformation, iontophoresis, topology, friction, trans epidermal water loss (TEWL), optical and heat dissipation. The means for measuring skin moisture content is then used to obtain at least two measured moisture content values from a consumer. In one example, measurement of the moisture content of a consumer is made for at least two areas on the skin, a control area that is in equilibrium with the environment and a test area. In one example, the control area may include the face, neck, ears, arms, hands, legs, feet, abdomen, back and groin, while the test area may include, for example, the face, neck, chest, back, arms, hands, feet, legs and scalp. However, several such measurements at various positions on the test area can be produced. In one example, the means for measuring skin moisture content is used to obtain one or more additional measured moisture content values on the test area. As each measured moisture content value is captured by the measurement means it is displayed in a suitable manner, as described above.

Next, the at least two measured moisture content values are compared to each other in order to obtain a measured moisture content differential. For example, the moisture content value for the control area (m1) is compared to the moisture content value for the test area (m2), to obtain a measured moisture content differential (m2−m1) or (m1−m2). In one embodiment, the measurement means is electrically coupled through a cable to a processing system, such as a conventional PC or laptop computer. In another embodiment, the measurement means is electrically coupled to a programmable integrated circuit. In yet another embodiment, the measurement means is electrically coupled to a computer wirelessly. The processing system is operable to convert the measured moisture content values generated by the measurement means into a measured moisture content differential. In another embodiment, the measured moisture content differential may be determined manually using the formula (m2−m1) or (m1−m2).

If additional test area moisture values are obtained, all of these values are compared to one another and the measurement with the largest value, or lowest value or mean value is then compared with the moisture content value for the control area. The result of this comparison is a measured moisture content differential profile. In one embodiment, a measurement differential from about 0 MHz to about 0.05 MHz, represents a healthy skin condition (high moisture level); a measurement differential from about 0.05 MHz to about 0.5 MHz represents a moderate skin condition (medium moisture level); and a measurement differential greater than about 0.5 MHz represents an unhealthy skin condition (low moisture level).

Next, the moisture content differential is correlated to a skin moisture value, in the same manner as described above with respect to the scalp moisture value. This skin moisture value is then used to assist in developing the personal care regimen for the consumer. In one example, the personal care regimen includes recommending at least one personal care product that will be effective for improving the skin health of a consumer, dandruff, flaking, dryness, tightness, itch, irritation and redness. Exemplary personal care products may include, without limitation, skin care products/devices, such as moisturizers, dry skin lotion, anti-rash products, anti-itch products; hair care products/devices, such as shampoos, conditioners, leave-on treatments, styling aids; baby care products/devices, such as diapers, diaper rash lotion, dry skin lotion, anti-itch lotion; and adult care products/devices, such as adult incontinence pads.

### EXAMPLES

The following examples are given solely for the purposes of illustration and are not to be construed as limitations of the present invention, as many variations of the invention are possible without departing from the spirit and scope of the invention.

### Example 1

Scalp Moisture Content and Dandruff

To determine a correlation between moisture content of scalp and dandruff, the scalp moisture content of consumers was measured using an electronic device comprising an impedance sensor as described above. The scalp moisture content for each consumer was determined as follows: One reading was taken from the forehead; and two readings were taken from the scalp. The measured moisture content values were then converted into a measured moisture content differential by the device. In addition, each consumer was assigned an Adherent Scalp Flaking Score (“ASFS”). The ASFS for each consumer was determined by having a qualified grader examine an octant of the consumer's scalp and then assigning a flake grade to that octant. Exemplary results are shown below Table II.

TABLE II

| | Measured Signal | | |
|---|---|---|---|
| Number of | Difference between | ASFS Octant | |
| Consumers | m1 and m2 (KHz) | Grade | Scalp Condition |
| 61 | 395.164 | 8-10 | Severe Flaking |
| 235 | 308.766 | 4-6 | Moderate Flaking |
| 169 | 230.527 | 0-2 | No Flaking |

### Example 2

Method of Measuring Scalp Moisture Content of a Consumer

In one embodiment of the present invention, a consumer enters a retail location to shop for hair care products. While shopping, the consumer is greeted by a beauty counselor or hair care consultant, who asks consumer to provide some personal information about hair and scalp health conditions and/or preferences. The consumer is invited to sit at a computer terminal where she views and completes the questions of the questionnaire about, for example, benefits that are most important, whether or not she likes a cool and tingling feeling while washing hair and brand of hair care product currently being used. The consumer answers the questions by selecting one of multiple possible answers. The consumer then views information on the importance of scalp moisturization and signs of dandruff. Next, the consumer's scalp is tested using a moisture meter to obtain objective measurements on the moisture content of the scalp. A first reading is taken from the forehead of the consumer. A second reading is then taken from the scalp. The measurements from each reading are used with the personal information to recommend the appropriate hair care product for the consumer based on her individual needs and conditions. This information is then provided to the consumer by the beauty counselor or hair care consultant in the form of a product picture on the computer screen. As the beauty counselor or hair care consultant provides the details of the product recommendation to the consumer, the beauty counselor or hair care consultant may also provide the consumer with brochures or other educational information for her own individualized needs. The products recommended to the consumer in order to improve the health of the consumer's hair and scalp are available for her purchase in the retail location.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for measuring scalp moisture content of a consumer, said method comprising the steps of:
a) collecting input information from said consumer regarding a plurality of characteristics associated with scalp condition of said consumer;
b) providing a moisture meter for measuring said scalp moisture content;
c) using said moisture meter to obtain at least one measured moisture content value from a position on scalp of said consumer;
d) using said input information and said at least one measured moisture content value to identify at least one hair care product for said consumer; and
e) providing said consumer with information regarding the utilization of said at least one hair care product.

2. The method according to claim 1, wherein the step of recommending at least one hair care product includes an automated data analysis.

3. The method according to claim 2, wherein the automated data analysis is performed using software.

4. The method according to claim 1, wherein said input information is obtained from said consumer by questioning means.

5. The method according to claim 4, wherein said questioning means is an interview in a retail location.

6. The method according to claim 1, wherein said moisture meter comprises an impedance sensor.

7. The method according to claim 1, further comprising the step of: obtaining a second measured moisture content value at a position off said scalp.

8. The method according to claim 7, further comprising the steps of: comparing said first measured moisture content value with said second measured moisture content value to obtain a measured moisture content differential; and correlating said measured moisture content differential to a scalp moisture value for said scalp.

9. The method according to claim 1, wherein said at least one hair care product is selected from the group consisting of shampoos, conditioners, oils, colorants, and styling compositions.

10. The method according to claim 1, wherein said information regarding said at least one hair care product is provided to said consumer through a written report.

11. The method according to claim 2, wherein said information regarding said at least one hair care product is provided to said consumer by means of graphical interface depicting the recommended at least one hair care product.

12. The method according to claim 11, wherein said graphical interface is outputted to an imaging device selected from the group consisting of a computer screen, television monitor, light radiating display and print materials.

13. A method for providing a personal care regimen to a consumer for at least one desired benefit toward an improved skin condition, said method comprising the steps of:
a) providing a means for measuring skin moisture content;
b) using said means for measuring skin moisture content to obtain at least two measured moisture content values from said consumer;
c) using said moisture content values to develop said personal care regimen for said consumer; and
d) providing said personal care regimen to said consumer thereby allowing said consumer to use said personal care regimen in a manner to provide optimum health for said skin of said consumer.

14. The method according to claim 13, wherein said personal care regimen comprises recommending at least one personal care product that will be effective for said consumer in improving health for said skin of said consumer.

15. The method according to claim 13, wherein said steps are performed in a retail location.

16. The method according to claim 13, wherein said personal care products are selected from the group consisting of skin care products, hair care products, baby care products and adult care products.

17. The method according to claim 13, wherein step (b) comprises obtaining a first measured moisture content value from a control area that is in equilibrium with environment and a second measured moisture content value at a position on a test area.

18. The method according to claim 17, further comprising the steps of: comparing said first measured moisture content value with said second measured moisture content value to obtain a measured moisture content differential; and correlating said measured moisture content differential to a skin moisture value for said skin.

19. A method for measuring scalp moisture content of a consumer in a retail location, said method comprising the steps of:
a) requesting input information from a consumer regarding said-consumer's hair care preferences, said request of said information being accomplished by questioning means;
b) using a moisture meter to obtain at least one measured moisture content value at a position off said scalp and at least one measured moisture content value at a position on said scalp;
c) analyzing said moisture content values to obtain a scalp moisture value for said scalp;
d) providing said consumer with information regarding said scalp moisture value; and
e) recommending to said consumer at least one product to improve scalp condition.

20. The method according to claim 19, wherein said area outside scalp location is selected from the group consisting of forehead skin, cheek, chink, ear and arm.
